(19) United States
(12) Patent Application Publication
(10) Pub. No.: US 2002/0107185 A1
(43) Pub. Date: Aug. 8, 2002

(54) Human fibrotic responses to surgery and methods of treatment

(76) Inventor: E Martin Spencer, San Francisco, CA (US)

Correspondence Address: E Martin Spencer, 505 Ortega Street, San Francisco, CA, 94122, US

(21) Appl. No.: 09/734,223

(22) Filed: Dec. 9, 2000

(51) Int. Cl. A61K 38/18; C07K 14/475

(52) U.S. Cl. 514/12; 530/399

(57) ABSTRACT

This invention is a method for selectively preventing an unwanted fibrotic response in humans after surgery or injury using therapeutic amounts of human insulin-like growth factor binding protein-4 (IGFBP-4) that has been modified to resist the enzymatic action of the natural IGFBP-4 protease in tissues. Surgical procedures in the abdomen, pelvis, thorax, and spinal column frequently initiate the formation of fibrous adhesions between organs. Collagen is the principal constituent of fibrous adhesions. These abnormal connections may interfere with the normal function of the organs involved, cause pain, and require additional surgical procedures, which frequently induces more adhesion formation. Surgical incisions and burns may directly initiate a process in tissues that stimulates excess fibrous tissue and results in hypertrophic scars or keloids that are disfiguring and/or interfere with function. At times a decreased fibrotic response is required by plastic surgeons for cosmetic reasons and by ophthalmologists for glaucoma patients to maintain patency of holes created in the sclera (sclerectomy) to filter fluid from the anterior chamber of the eye. Insulin-like growth factor-I (IGF-I) is one of the components that stimulates the adhesion process and wound repair. Lowering the level of IGF-I in the body significantly impairs wound healing and the synthesis of collagen. Lowering the level of IGF-I in the specific site of the body at which adhesions are undesirable significantly impairs the formation of adhesions and decreases the fibrotic response in wounds. IGFBP-4 inhibits the formation of fibrous tissue by binding and inactivating IGF-I. The protease resistant, modified IGFBP-4 is superior to the natural IGFBP-4 for this purpose.

### FIELD OF THE INVENTION

This invention relates to the fibrotic response in humans resulting from surgery or trauma and methods to inhibit this response.

### BACKGROUND

### Wound Healing

Normal wound healing consists of a coordinated series of reactions that restore the integrity of the tissue. The stages are: Hemostasis, inflammation, proliferation, matrix formation, and remodeling. Hunt T. K., “Wound healing: Disorders of repair, in” Fundamentals of Wound Management in Surgery, (Chirurgeocom 1976). After hemostasis is achieved, granulocytes appear followed by macrophages. Macrophages are critical to healing and direct the subsequent events by the production of cytokines, polypeptides and proteins that regulate cellular functions. Fibroblasts proliferate into the wound and synthesize collagen (fibrous tissue), the major component of the matrix of the wound. This is followed closely by angiogenesis, which is the proliferation and in-growth of blood vessels. Subsequently, in the case of cutaneous wounds, epithelial cells on the edge proliferate and cover the wound. The final stage is remodeling which includes modification of the collagen tissue and contraction of the wound scar. IGF-I stimulates most of the phases of wound healing, especially the activity of macrophages and fibroblasts, which proliferate and synthesize collagen, elastin and proteoglycans. Spencer, et al., “Somatomedins: Do they play a pivotal role in wound healing?,” Growth Factors and Other Aspects of Wound Healing: Biological and Clinical Implications, 103-116, (1988); Steenfos, et al., “Insulin-like Growth Factor 1 has a Major Role in Wound Healing,” Surgical Forum, 68-70, (1989); Mueller, et al., “The role of IGF-I and IGFBP-3 in wound healing,” in Modern Concepts of Insulin-like Growth Factors, ed. Spencer E. M. (Elsevier 1991) pp. 185-192; Karey K. P. and Sirbasku D., “Human platelet-derived mitogens. II. Subcellular localization of insulinlike growth factor I to the alpha-granule and release in response to thrombin,” Blood, 74, 1093-100, (1989); Suh, et al., “Insulin-like growth factor-I reverses the impairment of wound healing induced by corticosteroids in rats,” Endocrinology, 131, 2399-403, (1992); Mueller, et al., “The effect of insulin-like growth factor I on wound healing variables and macrophages in rats,” Archives Of Surgery, 129, 262-5, (1994).

### Adhesions

Etiology

Adhesions are defined as abnormal fibrous connections between organs. They consist principally of collagen.

Adhesive disease may stem from a number of causes, however, surgical invasion of the body cavities remains one of the foremost. They are the result of an inappropriate but heretofore unavoidable wound healing response, which leads to firm fibrous connections between organs where none existed previously. Postoperative intra-peritoneal adhesions begin to form as early as a few days after surgery and persist indefinitely.

Pathophysiology and Therapy

Because adhesions distort normal anatomical relationships, postoperative adhesions may be symptomatic. Peritoneal adhesions may cause chronic pain, small bowel obstruction, intestinal ischemia, infertility and increased complications upon subsequent surgery. Hunt T. K. and Gimbel M. L., “Postoperative adhesive disease,” Perspectives in Colon and Rectal Surgery, 11, 83-91, (1998). More than half of small bowel obstructions are caused by peritoneal adhesions. They account for a significant percentage of early postoperative obstructions in the four weeks following laparotomy. In long-term studies, about 5% of patients who undergo laparotomy develop obstruction secondary to adhesions. Landercasper, et al., “Long-term outcome after hospitalization for small bowel obstruction,” Archives of Surgery, 128, 765-778, (1993). Any surgical procedure in the abdomen may result in adhesions, but operations in the lower abdomen, such as appendectomy or hysterectomy, are frequently the cause.

Numerous approaches to preventing adhesion formation in body cavities have been tried with variable success. Beneficial practices include gentle, atraumatic surgery, avoidance of powdered gloves, and meticulous anastomoses that minimize leakage and infection. Diamond M. P., “Surgical aspects of infertility,” Gynecology and Obstetrics: Endocrinology, Infertility, and Genetics, (1991); diZerega G. S., “Contemporary adhesion prevention,” Fertil Steril, 61, 219-235, (1994); Sannella N. A., “Early and late obstruction of the small bowel after abdominoperineal resection,” Am J Surg, 130, 270-2, (1975). Many other attempts to reduce adhesions have been tried and discarded, including instillation of both viscous liquids and thin electrolyte solutions. diZerega, “Contemporary adhesion prevention,” Fertil Steril, 61, 219-235, (1994); Adhesion Study Group,“. Reduction of postoperative pelvic adhesions with intraperitoneal 32% dextran 70: a prospective, randomized clinical trial.,” Fertil. Steril, 40, 612-9, (1983); Larsson, et al., “Effect of intraperitoneal instillation of 32% dextran 70 on postoperative adhesion formation after tubal surgery,”. Acta Obstet Gynecol Scand, 54, 437-41, (1985); Jansen, “Failure of intraperitoneal adjuncts to improve the outcome of pelvic operations in young women,” Am J Obstet Gynecol, 153, 363-371, (1984). Systemic agents such as anti-inflammatory steroids have had disastrous results. diZerega G. S., “Contemporary adhesion prevention,” Fertil Steril, 61, 219-235, (1994); Milligan and Raftery, “Observations on the pathogenesis of peritoneal adhesions: a light and electron microscopical study,” Br J Surg, 61, 274-80, (1974). Early results from the use of membrane barriers are premature and not promising for prevention of bowel obstructions. Barriers are associated with their own drawbacks such as possible increased risk of infection. Diamond, et al., “Interceed (TC7) absorbable adhesion barrier.,” Infertility and Reproductive Medicine Clinics of North America, 5, 485-508, (1994); Diamond M. P. and Seprafilm Adhesions Study Group, “Seprafilm (HAL-F) reduces postoperative adhesions: Initial results of a multicenter gynecologic clinical study,” The 3rd International Congress on Pelvic Surgery and Adhesion Prevention., (1996); Becker, et al., “Prevention of postoperative abdominal adhesions by a sodium hyaluronate-based bioresorbable membrane: a prospective, randomized, double-blind multicenter study [see comments],” J Am Coll Surg, 183, 297-306, (1996); DeCherney A. H. and diZerega G. S., “Clinical problem of intraperitoneal postsurgical adhesion formation following general surgery and the use of adhesion prevention barriers.,” Surg Clin North Am, 77, 671-88, (1997); Haney A. F. and Doty E., “Murine peritoneal injury and de novo adhesion formation caused by oxidized-regenerated cellulose Interceed [TC7] but not expanded polytetrafluoroethylene Gore-Tex Surgical Membrane,” Fertil Steril, 57, 202-8, (1992). The ideal agent is one that could be applied to a wide region with minimal effect on critical areas and incisions, and no systemic effects. To date no one has successfully addressed blocking adhesion formation by interfering with the stimulatory action of IGF-I on fibrous tissue formation.

### Hypertrophic Scars and Keloids

Hypertrophic scars and keloids are caused by an exaggerated wound healing response that deposits and continues to deposit over long periods of time unsightly, excess scar tissue. Hunt T. K., “Wound healing: Disorders of repair,” Fundamentals of Wound Management in Surgery, (Chirurgecom 1976). In the case of hypertrophic scars the excess collagen is limited to the incision line or the area involved in a 3^{rd} degree burn. Keloids are tumorous masses of collagen initiated by an incision (injury) but which extend widely beyond the margins of the incision. Keloids are typically found in the upper part of the body and certain races have an increased incidence. Therapy of these conditions involves the injection of glucocorticoids, in some cases administered after careful surgical excision, and prolonged application of impermeable plastic dressings. However, frequently therapy is ineffective. To date no one has successfully addressed blocking collagen synthesis by interfering with the stimulatory action of IGF-I.

### Inhibition of Normal Wound Healing

Plastic surgeons frequently desire to limit the normal amount of fibrous (scar) tissue in incisions on exposed parts of the body for cosmetic reasons. In treating patients with glaucoma ophthalmologists would like to maintain the patency of holes created in the sclera (sclerectomy) to drain fluid from the anterior chamber the eye. Frequently the normal wound healing response results in closure of this opening and failure of the therapy. Mitomycin is placed at the time of surgery about the opening to inhibit the proliferation and function of the fibroblasts, which are responsible for closing the opening. However, the dose range is critical, too low being ineffective and too much causing low intra-ocular pressure, a serious complication. In addition, infection occurs in approximately 1% of patients. After surgery mitomycin can not be used again if the scleral hole begins to close. In this situation 5-fluoruracil can be injected about the hole to slow this process by inhibiting cell growth, but frequently this is ineffective. To date no one has successfully addressed blocking the wound healing response in these two conditions by interfering with the action of IGF-I on collagen synthesis.

### Insulin-like Growth Factor-I

Insulin-like growth factor-I (also referred to as somatomedin-C and IGF-I) is an anabolic, growth-promoting polypeptide of 7,648 daltons. IGF-I is the mediator of the growth-promoting action of growth hormone. Schlechter, et al., “Evidence suggesting that the direct growth-promoting effect of growth hormone on cartilage in vivo is mediated by local production of somatomedin,” Proceedings Of The National Academy Of Sciences Of The United States Of America, 83, 7932-4, (1986). It stimulates the proliferation and differentiation of a wide variety of cells and promotes multiple cell and organ functions, frequently working in concert with other hormones and factors. Spencer E. M., “Somatomedins,” in Basic and Clinical Endocrinology, (Appleton and Lange 1991) pp. 89-99; Spencer E. M., “Directions for research into the insulin-like growth factor system as the millennium approaches; Closing remarks to the IVth IGF Symposium,” Excerpta Medica, International Congress Series 1151, (1998). IGF-I has a prominent action on wound healing. Depleting wounds of IGF-I decreases healing. Steenfos, et al., “Insulin-like Growth Factor 1 has a Major Role in Wound Healing,” Surgical Forum, 68-70, (1989). Augmenting wound IGF-I promotes healing. Sommer, et al., “Molecular genetics and actions of recombinant insulin-like growth factor binding protein-3,” in Modern Concepts of Insulin-like Growth Factors, ed. Spencer, E. M. (Elsevier 1991) pp. 715-728; Suh, et al., “Insulin-like growth factor-I reverses the impairment of wound healing induced by corticosteroids in rats,” Endocrinology, 131, 2399-403, (1992). IGF-I exerts its action throughout the tissue repair process, but is especially important in stimulating fibroblast proliferation and synthesis of collagen.

### Insulin-like Growth Factor Binding Protein-4

IGF-I action is regulated at the tissue level by a class of 6 homologous, circulating, specific IGF binding proteins (IGFBPs). Martin J. L. and Baxter R. C., “IGF Binding Proteins as Modulators of IGF Action,” in The IGF System: Molecular Biology, Physiology and Clinical Applications, (Humana 1999) pp. 227-255. After association with IGF-1, the action of IGF-I is generally inhibited by IGFBPs, but under certain experimental conditions stimulation is achieved. Sommer, et al., “Molecular genetics and actions of recombinant insulin-like growth factor binding protein-3,” in Modern Concepts of Insulin-like Growth Factors, ed. Spencer E. M. (Elsevier 1991) pp. 715-728; Martin J. L. and Baxter R. C., “IGF Binding Proteins as Modulators of IGF Action,” in The IGF System: Molecular Biology, Physiology and Clinical Applications, (Humana 1999) pp. 227-255. However, it is generally considered that IGFBP-4 is the only purely inhibitory IGFBP. Shimasaki, et al., “Isolation and molecular characterization of three novel insulin-like growth factor binding proteins (IGFBP-4, 5 and 6),” Modern Concepts of Insulin-like Growth Factors, 343-358, (1991); Fowlkes and Freemark, “Evidence for a novel insulin-like growth factor (IGF)-dependent prolease regulating IGF-binding protein-4 in dermal fibroblasts,” Endocrinology, 131, 2071-2076, (1992); Byun, et al., “Studies on human pregnancy-induced insulin-like growth factor (IGF)-binding protein-4 proteases in serum: determination of IGF-II dependency and localization of cleavage site,” J Clin Endo Metab, 85, 373-381, (2000); Chelius, et al., “Expression, purification and characterization of the structure and disulfide linkages of insulin-like growth factor binding protein-4 (IGFBP-4),” J Endocrinology, Submitted, (2000).

The action of IGFBP-4 is inhibited by an IGFBP-4 specific protease, pregnancy-associated plasma protein-A (PAPP-A), which abrogates IGF-I binding by specifically cleaving the IGFBP-4 molecule into 2 fragments of similar size. Lawrence, et al., “The insulin-like growth factor (IGF)-dependent IGF binding protein-4 protease secreted by human fibroblasts is pregnancy-associated plasma protein-A,” Proc. Natl. Acad. Sci. U.S.A., 96, 3149-3153, (1999). The cleavage site is between Met¹³¹-Lys¹³² for rat IGFBP-4 corresponding to Met¹³⁵-Lys¹³⁶ for human IGFBP-4 (vide infra).

The inhibitory effects of IGFBP-4 on IGF-I action may play an important role in local proliferative responses of IGFs such as in bone remodeling, human reproduction, atherosclerotic plaque development, as well as wound healing. Lawrence, et al., “The insulin-like growth factor (IGF)-dependent IGF binding protein-4 protease secreted by human fibroblasts is pregnancy-assoicated plasma protein-A,” Proc. Natl. Acad. Sci. U.S.A., 96, 3149-3153, (1999). At present IGFBP-4 is not approved for therapeutic use.

IGFBP-4 may be useful in vivo to inhibit the formation of postoperative adhesions after surgery on the abdominal, thoracic and spinal cavities. IGFBP-4 may also be useful to inhibit the occurrence or progression of hypertrophic scars and keloids. IGFBP-4 may also be useful to depress the normal wound healing response to yield smaller scars and to maintain the patency of holes in the sclera for the treatment of glaucoma.

### DISCLOSURE OF THE INVENTION

In this description, the following terms are employed:

Abbreviations, single letter

C. Cytidine, one of the four nucleotides that make up DNA

G. Guanidine, one of the four nucleotides that make up DNA

Clones

Genetically identical copies

Cloned into

Inserted into

DpnI

A restriction enzyme that digests parental, supercoiled double-stranded DNA

dNTP

A mixture of the 4 nucleotide triphosphates

Electrospray mass spectrometry

A method for determining the exact mass of proteins and other compounds

Epicurian Coli XL 1-Blue supercompetent cells

Bacterial cells that have been rendered susceptible to the introduction of exogenous plasmids

Expression vector

A plasmid specifically constructed to instruct the host to synthesize the protein that its DNA specifies

Glycyl-IGFBP-4

IGFBP-4 that has been expressed (produced) by recombinant DNA methodology which has an additional glycine residue at the N-terminus

Hypophysectomy

A surgical procedure that removes the pituitary gland and the source of growth hormone, thyroid stimulating hormone, adrenocorticotropic hormone and gonadotropins (hormones that stimulate the gonads)

HPLC

High performance liquid chromatography. A method for isolating chemical compounds

Kb

A thousand bases

Laparotomy

A surgical procedure that opens the abdomen

Melting temperature (Tₘ)

The midpoint of the temperature range at which two complementary strands of DNA separate

Mutant

A protein with an altered amino acid sequence from the naturally occurring variety

NZY+broth

A medium for growing Epicurean Coli bacteria used in the Stratogene kit.

Oligonucleotide

A polymer composed of nucleotides of unidentified length generally under a few hundred

Parietal peritoneum

A smooth membrane that lines the wall of the abdominal and pelvic cavities

PCR

Polymerase chain reaction. A series of chemical reactions performed in repeated cycles to increase the number of copies of the starting nucleic acid material

pET32

A commonly used expression vector. It is a plasmid that is introduced into a host bacterial cell that directs the synthesis of a protein specified in its DNA sequence

PfuTurbo DNA polymerase

An enzyme that catalyzes the growth of the DNA chain during the PCR reaction

Plasmid

A circular piece of DNA used to transfer genetic material to host cells

Primer

An oligonucleotide of usually greater than 20 nucleotides with a high enough sequence similarity to a corresponding area on a strand of DNA so that it will bind to that area

Restriction enzymes

Enzymes that cut DNA at specific sites

SDS PAGE

Sodium dodecyl sulfate polyacrylamide gel electrophoresis. A method of separating proteins by their molecular weight.

Site-directed mutagenesis

A technique to change the sequence of a gene in a precise locus thereby changing the amino acid specified at that position

Somatomedin-C

An earlier name for insulin-like growth factor-I, IGF-I

Template

The DNA to which the primers bind and which furnishes the intervening sequence to be copied in the PCR reaction

Thyroxine

Thyroid hormone

Transformation

A change in a bacteria induced by the introduction of foreign DNA

Uterine horn

One of the two lateral parts of the ‘Y’ shaped rat uterus.

The invention solves the problems of postoperative adhesion formation, excess wound scarring, and decreasing the normal wound healing response.

In experimental studies the formation of intraperitoneal adhesions was investigated using a rat uterine horn model. At laparotomy small areas on the lateral side of one uterine horn and the adjacent parietal peritoneum were abraded. The two areas were loosely approximated by a suture distal to the abraded areas and the abdomen was closed. On postoperative day 10 postmortem laparotomies were performed and uterine-peritoneum adhesions were scored on a 0 to 3+ system by blinded observers. In one series of experiments the level of IGF-I was lowered in 15 rats by hypophysectomy, which removes the source of growth hormone, the major stimulator of IGF-I synthesis. The attendant losses of thyroxine and adrenal cortical steroids in these rats were replaced. When studied after 10 days and compared to placebo, the IGF-I deficient rats had significantly less severe adhesions. In another series of experiments uterine horn abrasions were created in rats with normal IGF-I levels. IGF-I action in the abdominal cavity was specifically blocked by the twice-daily intraperitoneal administration of 3 ml of recombinant rat glycyl-IGFBP-4 (24 ug/ml). When studied after 10 days and compared to placebo, the IGFBP-4 treated rats had significantly less severe adhesions. No systemic effects were noted and the plasma levels of IGFBP-4 and IGF-I were unaltered compared to controls.

Since decreasing IGF-I levels by two different means resulted in less severe adhesion formation, it is likely that IGF-I stimulates adhesion formation and that blocking its action decreases adhesion formation. Since collagen tissue is the major part of adhesions, it is also likely that IGFBP-4 would inhibit collagen tissue formation in hypertrophic scars and keloids as well as slow the healing of surgically created incisions and holes created in the sclera.

To improve the effectiveness of IGFBP-4 experimental studies were done to determine the site at which the IGFBP-4 specific protease, PAPP-A, cleaves and inactivates IGFBP-4 so that IGFBP-4 could be modified to become resistant to PAPP-A. Conover, et al., “Posttranslational regulation of insulin-like growth factor binding protein-4 in normal and transformed human fibroblasts,” J Clin Inves, 91, 1129-1137, (1993); Fowlkes and Freemark, “Evidence for a novel insulin-like growth factor (IGF)-dependent protease regulating IGF-binding protein-4 in dermal fibroblasts,” Endocrinology, 131, 2071-2076, (1992); Conover, et al., “Cleavage analysis of insulin-like growth factor (IGF)-dependent binding protein-4 proteolysis and expression of protease-resistant IGF-binding protein-4 mutants,” J Biol Chem, 270, 4395-4000, (1995). Other IGFBP-4 proteases have been found in neuroblastoma conditioned medium and pregnancy sera, but their relationship to PAPP-A has not been established, nor has a single unique cleavage site been defined for PAPP-A or any other IGFBP-4 protease. Chernausek, et al., “Proteolytic cleavage of insulin-like growth factor binding protein 4 (IGFBP-4),” J Biol Chem, 270, 11377-11382, (1995); Byun, et al., “Studies on human pregnancy-induced insulin-like growth factor (IGF)-binding protein-4 proteases in serum: determination of IGF-II dependency and localization of cleavage site,” J Clin Endo Metab, 85, 373-381, (2000). To demonstrate specific enzymatic cleavage the following must be established. Two fragments, which contain the entire amino acid sequence, must be quantitatively isolated and shown to possess the original N- and C-terminal residues, plus new N- and C-terminal residues contiguous in the amino acid sequence. To fulfill these criteria our study used recombinant rat glycyl-IGFBP-4 that had been expressed in E. Coli and purified. Lu X. B. and Spencer E. M., “Expression and purification of human and rat IGF binding proteins-4, rat IGF binding protein-3 in E coli,” 80th Annual Meeting The Endocrine Society, 317, (1998). IGFBP-4 (1 mg) was cleaved by incubation with 8 ml of adult human fibroblast conditioned medium, the source of PAPP-A, containing 280 ug of IGF-I for 72 hours at 37 C. Lawrence, et al., “The insulin-like growth factor (IGF)-dependent IGF binding protein-4 protease secreted by human fibroblasts is pregnancy-associated plasma protein-A,” Proc. Natl. Acad. Sci. U.S.A., 96, 3149-3153, (1999). Fragments were separated by reverse phase HPLC on a C₄ column (Vydac) using a 25-30% gradient of acetonitrile in 0.1% trifluoroacetic acid. The fractions containing polypeptides were identified by electrospray mass spectrometry. The cleavage quantitatively produced only two fragments of molecular weights 14,429.7 and 11,322.0 daltons. The larger fragment corresponded exactly to the N-terminal region of glycyl-IGFBP-4 ending with methionine at position 131. The smaller fragment corresponded exactly to the C-terminal region of rat glycyl-IGFBP-4 beginning with lysine at position 132. The cleavage was blocked by the addition of a specific antibody to PAPP-A in the incubation medium.

This invention provides a method for increasing the effectiveness of IGFBP-4 by expressing a mutant with an altered cleavage site rendering IGFBP-4 resistant to PAPP-A. Two types of mutants can be created using site-directed mutagenesis. 1) Methionine 135 and lysine 136 at the cleavage site of human IGFBP-4 can be mutated to amino acids which are resistant to PAPP-A, such as aspartic-aspartic, proline-proline or alanine-alanine, but not excluding other amino acids. Additional flanking residues can be mutated if necessary to achieve full resistance to enzymatic activity. 2) As an alternative a deletion mutant encompassing the cleavage site and several flanking residues on both sides can be constructed.

The procedures for accomplishing site-directed mutagenesis are now standard. The Stratagene QuikChange TM Site-Directed Mutagenesis Kit can be use to switch amino acids, delete or add single or multiple amino acids.

i) The oligonucleotide primers will be between 25 and 45 bases in length, and the melting temperature (Tₘ) of the primers will be greater than or equal to 78° C. using the formula:

Tₘ=81.5+0.41(%GC)−675/N−%mismatch,

where N is the primer length in base pairs.

ii) The desired mutation (deletion or insertion) will be in the middle of the primer with ˜10-15 bases of correct sequence on both sides,

iii) The primers optimally should have a minimum GC content of 40% and should terminate in one or more C or G bases.

iv) A computer program will evaluate each primer for its suitability.

v) Primers will be purified according to the recommended protocol.

Site-directed mutagenesis will be carried out as follows: 125 ng of the oligonucleotide primers #1 and #2 encoding the mutant residues, 5 μl of 10 times concentrated reaction buffer, and 1 μl dNTP mixture are added to 5, 10, 20 and 50 ng double stranded human IGFBP-4 plasmid DNA template and filled up to 50 μl with double-distilled H₂O. 1 μl PfuTurbo DNA polymerase (2.5 U/μl) is added to the reaction mixture, which is then overlaid with 30 μl of mineral oil and PCR cycled to produce mutant copies of the plasmid as follows:

| Segment | Cycles | Temperature | Time |
|---|---|---|---|
| 1 | 1 | 95° C. | 30 seconds |
| 2 | 12-18 | 95° C. | 30 seconds |
| | | 55° C. | 1 minute |
| | | 68° C. | 2 minutes/kb of plasmid |

where the number of cycles for point mutations is: 16 for single amino acid changes and 18 for multiple amino acid deletions or insertions.

Following the PCR cycling, the samples are placed on ice for 2 minutes to cool the reaction to 37° C. 1 μl of the DpnI restriction enzyme (10 U/μl) is added directly to each amplification reaction below the mineral oil layer and incubated for 1 hour at 37° C. to digest the original parental (nonmutated) supercoiled dsDNA leaving only the mutated plasmids.

Epicurian coli XL1 -Blue supercompetent cells are gently thawed on ice and a 50 μl aliquot for each sample reaction is transformed to a prechilled polypropylene tube. 1 μl of the Dpn-I treated DNA from each sample reaction is transferred to a separate aliquot of supercompetent cells. The transformation mix is gently mixed and incubated on ice for 30 minutes. The cells are heat-shocked for 45 seconds at 42° C. and then placed on ice for 2 minutes. 0.5 ml of NZY+ broth, preheated to 42° C., is added and the mixture is incubated for 1 hour at 37° C. with shaking. The entire volume of each sample transformation is plated on agar pates containing ampicillin and incubated overnight at 37° C. Positive clones will be identified by sequencing and cloned into the expression vector pET32.

Expression and purification of the mutants will be carried out as described for IGFBP-4. Lu X. B. and Spencer E. M., “Expression and purification of human and rat IGF binding proteins-4, rat IGF binding protein-3 in E coli,” 80th Annual Meeting The Endocrine Society, 317, (1998). Mutants will be assayed by SDS PAGE and mass spectroscopy then tested for resistance to PAPP-A.

The binding affinity of the protease resistant mutant IGFBP-4 for IGF-I will be measured by classic kinetic methods and calculated using GraphPad software. Taylor V. L. and Spencer E. M., “Identification of a plasma membrane receptor for insulin-like growth factor-3 (IGFBP-3) on human platelets,” J Endocrinology, in press, (2000). The biologic potency of the mutant IGFBP-4 in inhibiting IGF-I stimulation will be tested in at least two IGF-I-dependent systems in which IGF-I stimulates cellular proliferation. This would include such cells as osteoblasts, chicken embryo fibroblasts, and aortic smooth muscle cells. The mutant with essentially the same or higher binding affinity and IGF-I inhibition as authentic IGFBP-4 will be selected for therapeutic use.

### BEST MODE OF CARRYING OUT THE INVENTION

Apply protease resistant IGFBP-4 in a therapeutic concentration formulated as a paste in a suitable vehicle, such as fibrin, to the areas operated upon at the time of surgery. Inject protease resistant IGFBP-4 in a therapeutic concentration for a course to be determined into hypertrophic scars and keloids. Administer subconjunctival injections of protease resistant IGFBP-4 in a therapeutic concentration for a course to be determined around scleral holes.

What is claimed is:

1. A modified human IGFBP-4 that is resistant to the action of the IGFBP-4 protease, PAPP-A. A modification that occurs at the cleavage site of the IGFBP-4 protease and consists of substituting at least amino acid residues 135 methionine and 136 lysine with other amino acids. The modification may include substitution of up to an additional 5 residues flanking the cleavage site or a deletion involving the cleavage site plus up to 5 additional residues.

2. A method for preventing postoperative adhesion formation in the abdomen and pelvis of a patient comprised of local application to wounded surfaces intra-operatively of an effective amount of protease resistant insulin-like growth factor binding protein-4.

3. A method for preventing postoperative adhesion formation in the abdomen and pelvis of a patient comprised of local application to wounded surfaces intra-operatively of an effective amount of insulin-like growth factor binding protein-4.

4. A method for preventing postoperative adhesion formation in the abdomen and pelvis of a patient comprised of local application to wounded surfaces intra-operatively of an effective amount of a mimetic capable of blocking the action of insulin-like growth factor-I.

5. A method for preventing postoperative adhesion formation in the thoracic cavity or spinal column of a patient comprised of local application to wounded surfaces intra-operatively of an effective amount of protease resistant insulin-like growth factor binding protein-4.

6. A method for preventing postoperative adhesion formation in the thoracic cavity or spinal column of a patient comprised of local application to wounded surfaces intra-operatively an effective amount of insulin-like growth factor binding protein-4.

7. A method for preventing postoperative adhesion formation in the thoracic cavity or spinal column of a patient comprised of local application to wounded surfaces intra-operatively an effective amount of a mimetic capable of blocking the action of insulin-like growth factor- I.

8. A method for preventing hypertrophic scars and keloids comprised of local application to wounded surfaces intra-operatively an effective amount of protease resistant insulin-like growth factor binding protein-4.

9. A method for preventing hypertrophic scars and keloids comprised of local application to wounded surfaces intra-operatively an effective amount of insulin-like growth factor binding protein-4.

10. A method for preventing hypertrophic scars and keloids comprised of local application to wounded surfaces intra-operatively an effective amount of a mimetic capable of blocking the action of insulin-like growth factor-I.

11. A method for treating hypertrophic scars and keloids comprised of local applications or intra-lesional injections of an effective amount of protease resistant insulin-like growth factor binding protein-4 over a plurality of days.

12. A method for treating hypertrophic scars and keloids comprised of local applications or intra-lesional injections of an effective amount of insulin-like growth factor binding protein-4 over a plurality of days.

13. A method for treating hypertrophic scars and keloids comprised of local applications or intra-lesional injections of an effective amount of a mimetic capable of blocking the action of insulin-like growth factor-I over a plurality of days.

14. A method for decreasing scar formation and retarding the closure of holes in the sclera comprised of a local application at the time of surgery of an effective amount of protease resistant insulin-like growth factor binding protein-4.

15. A method for decreasing scar formation and retarding the closure of holes in the sclera comprised of a local application at the time of surgery of an effective amount of insulin-like growth factor binding protein-4.

16. A method for decreasing scar formation and retarding the closure of holes in the sclera comprised of a local application at the time of surgery of a mimetic capable of blocking the action of insulin-like growth factor-I.

17. A method for maintaining the patency of holes placed in the sclera to drain fluid from the anterior chamber of the eye of patients with glaucoma comprised of local injections around the opening of an effective amount of protease resistant insulin-like growth factor binding protein-4.

18. A method for maintaining the patency of holes placed in the sclera to drain fluid from the anterior chamber of the eye of patients with glaucoma comprised of local injections around the opening of an effective amount of insulin-like growth factor binding protein-4.

19. A method for maintaining the patency of holes placed in the sclera to drain fluid from the anterior chamber of the eye of patients with glaucoma comprised of local injections around the opening of an effective amount of a mimetic capable of binding insulin-like growth factor-I.
